(19) United States
(12) Patent Application Publication
(10) Pub. No.: US 2025/0326933 A1
(43) Pub. Date: Oct. 23, 2025

(54) SOLID COMPOSITION OF INDOCYANINE GREEN AND SODIUM FLUORESCEIN

(71) Applicant: ICROM Srl, Concorezzo (MB) (IT)

(72) Inventors: Pierfrancesco Morosini, Concorezzo (MB) (IT); Daniele De Zani, Concorezzo (MB) (IT)

(73) Assignee: ICROM Srl, Concorezzo (MB) (IT)

(21) Appl. No.: 18/546,065

(22) PCT Filed: Mar. 21, 2022
(86) PCT No.: PCT/IB2022/052553

§ 371 Date: Aug. 10, 2023

(30) Foreign Application Priority Data
Mar. 22, 2021 (IT) 102021000006794
Mar. 22, 2021 (IT) 102021000006809
Oct. 12, 2021 (IT) 102021000026075

(51) Int. Cl. C09B 67/22; A61K 49/00; C09B 67/42

(52) U.S. Cl. CPC C09B 67/0033; A61K 49/0034; A61K 49/0043; C09B 67/0092

(57) ABSTRACT

The present invention relates to a new solid state composition of the compound of formula (IA), indocyanine green, and the compound of formula (IB), sodium fluorescein, the use thereof in the diagnostic field and a kit containing such composition.

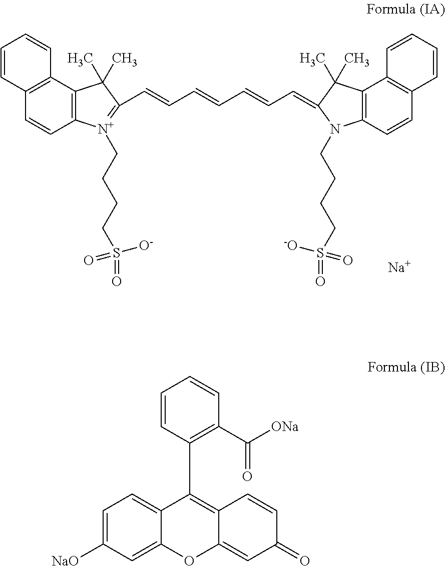

The present invention relates to a new solid state composition of the compound of formula (IA), indocyanine green, and the compound of formula (IB), sodium fluorescein, the use thereof in the diagnostic field and a kit containing such composition.

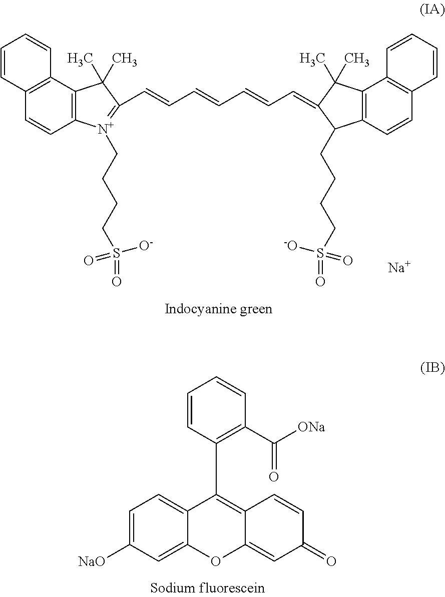

### FIELD OF THE INVENTION

The present invention relates to the field of contrast agents, in particular to those used in ophthalmology.

### STATE OF THE ART

Fluorescein angiography (FAG) is a useful tool for diagnosing many diseases of the retina. Fluorescein is normally administered as a sodium salt which at room temperature is presented as an odourless red-brown solid, which emits intense fluorescence in the range 520-530 nm (with a very characteristic yellow-green colour) when it is excited by ultraviolet rays at 254 nm and in the blue range (465-490 nm). Sodium fluorescein is on the market in a sterile aqueous solution in 5 ml or 2 ml vials, with various concentrations from 0.25 g/ml, 0.2 g/ml to 0.1 g/ml, differently in Europe or the USA.

Such a technique provides diagnostics and useful information for subsequent treatment enabling retinal and choroidal vascularization to be viewed. This test enables any unserved (ischemic) areas to be highlighted and any lesions caused by retinal neovascularization. The green-yellow fluorescence of the drug demarcates the vascular area of the retina and the iris.

Fluorescein is completely removed by the kidneys within 24-36 hours of intravenous administration.

More recently, Indocyanine Green Angiography (ICGA) has been developed. This dye was developed for photography by Kodak Research Laboratories in 1955 and was already approved for clinical use in 1956, but it took 10 years for it to be used in angiography and until the 1970s for it to be used in retinal angiography.

Indocyanine green of formula (IA), (ICG, 1H-benz[e]indole, 2-[7-[1,3-dihydro-1,1-dimethyl-3-(4-sulfobutyl)-2H-benz[e]indol-2-ylidene]-1,3,5-heptatrienyl]-1,1-dimethyl-3-(4-sulfobutyl) hydroxide, inner salt, sodium salt, CAS RN 3599-32-4) is sold in the solid state in the form of sterile freeze-dried powder containing 25 mg or 50 mg of indocyanine green in the presence of no more than 5% of sodium iodide. The amount that can be administered for ophthalmic angiography must not exceed 0.1-0.3 mg/kg of body weight as a bolus injection. The 25 mg dose is dissolved in 5 ml of water for injectable solutions and the 50 mg dose in 10 ml so that the 1 ml of reconstituted injectable solution contains 5 mg of indocyanine green.

The total daily dose in adults must be kept below 5 mg/kg of body weight. The maximum absorption and emission of indocyanine green are both around the infrared range, the maximum absorption at 800 nm and the maximum emission for measuring fluorescence at 830 nm. When dissolved in water, indocyanine green only shows a non-detectable decomposition for a few hours.

The presence of sodium iodide can cause severe anaphylactic reactions and must therefore only be used under the supervision of a physician.

Doses of up to 40 mg of indocyanine green dye can be used in 2 ml of sterile water for injectable preparations. A 5 ml bolus of normal saline solution must immediately follow the injection of the dye.

The indocyanine green molecule is the largest (molecular weight 775 Da compared to 332 Da for fluorescein) hence it bonds more strongly to the proteins in the plasma compared to fluorescein and is instead fluorescent in the infrared spectrum. It is therefore metabolised by the liver.

Initially ICG angiography was carried out using infraredinfrared photographic film. However, the poor sensitivity of the film along with the relatively weak fluorescence properties of the dye have led to this method being abandoned.

The strong bond of indocyanine green to plasma proteins causes slower metabolism compared to fluorescein and reduces the amount of fluorescence available for imaging. Digital video cameras were used to acquire images for ICG angiography.

Another approach to fluorescence angiography (using fluorescein or indocyanine green) is the scanning laser ophthalmoscope. This tool became part of common clinical practice because of its marketing over recent years. The advantages of scanning laser ophthalmoscopy include the possibility to use excitation light that scans the retina, enabling more intense excitation (thus providing a stronger emission signal) but using safe lighting levels. This is possible as the scanning range illuminates each point of the area of the retina for just 0.1-0.7 microseconds. Angiography with a scanning laser ophthalmoscope provides more accessible time information than static imaging systems as the real video enables imaging at speeds from 20 to 30 photograms per second.

Although ICG angiography may be advantageous in some cases of subretinal neovascularization and the identification of other disorders, one of its disadvantages is the long period of time required for angiography (45 minutes) and the need to obtain a second angiogram after obtaining an angiogram with fluorescein. The process for obtaining a fluorescein angiogram and an ICG angiogram takes a long time and requires 2 or 3 injections per patient for completing the study set. Furthermore, the angiograms are carried out at different times, making it difficult to understand whether the fluorescence leakage variations are due to differences in the properties of the dyes or the quality of the images.

To overcome these drawbacks, back in 1998 simultaneous angiographs were carried out with the joint administration of fluorescein and indocyanine green (W. R. Freeman et al., Arch. Ophthalmol., vol. 116, Apr. 1998, 455-463) using a small digital confocal scanning laser ophthalmoscope based on a PC to enable simultaneous imaging after a single intravenous injection of indocyanine green and fluorescein. Clinically, no severe adverse reactions were observed in a series of 169 simultaneous injections of indocyanine green and fluorescein. The necessary time for carrying out the entire study was considerably shorter than the initial time of a study on fluorescein, re-examining it and subsequently carrying out the study with indocyanine green.

Currently, the two dyes are injected simultaneously and a few seconds after the injection they reach retinal and choroidal level. Using a special instrument known as a fluorangiograph it is possible to photograph the back of the eye throughout all the steps of the passage of the dye and thus identify the presence of alterations such as: ischemic areas, neovascularizations, areas of diffusion of dye from the optical disc or from the retinal vessels (leakage), inflammatory foci, etc. FAG and ICGA are routine but invasive tests, therefore the renal function must be verified before carrying out the test as fluorescein is eliminated from the kidneys some hours after injection and it it necessary to be sure that the patient is not allergic to the contrast agents. The test is performed in general on an empty stomach or after breakfast or very light meals.

Nowadays, with the arrival of high resolution OCT and the modern OCT angiography, this invasive test can often be avoided. However, there are some conditions in which it is still fundamental and a multi-mode approach is necessary for achieving a correct diagnostic and therapeutic picture of the patient. The ophthalmologist must decide on a case-by-case basis which test(s) is/are most suitable for each individual patient.

The simultaneous administration of the two dyes takes place in practice by adding the content of the vial of aqueous solution of sodium fluorescein to the indocyanine green powder so as to obtain a sterile solution of the mixture of the two dyes without the presence of precipitates. It is clear how this preparation method can imply possible problems due to the need to withdraw a liquid solution containing sodium fluorescein with one syringe, add it to the sterile powder of indocyanine green, wait for the solution to form without having any precipitates and then withdraw the final solution again with the same syringe or with a different one.

Another problem may be comprised by the need to administer different quantities of dyes from those on the market and therefore to have to dispose of the mixture, which could not however be administered to another patient. This disposal can be expensive due to the high cost of indocyanine green.

Another problem comes from the instability of indocyanine green in aqueous solution for a prolonged period and therefore the impossibility to store the mixture.

The object of the present invention is a new composition in the solid state, obtained by the simultaneous freeze-drying of the two dyes, compounds of formula (IA) and (IB), wherein the quantity of the compound of formula (IA) is 2.5 mg, 5 mg, 10 mg, 25 mg, 50 mg or 100 mg and the quantity of the compound of formula (IB) is 0.1 g, 0.2 g, 0.4 g, 2.5 g, 2.0 g, 1.25 g or 1 g. The concentrations that can be obtained by dissolving the mixture in water are provided in the Experimental part.

The Applicant surprisingly found that the composition of the compounds of formula (IA) and (IB), when prepared through freeze-drying, is soluble in water and stable, even without the addition of NaI as took place in the state of the art for stabilizing the compound of formula (IA). The composition of the present invention can contain NaI in the quantities 0% ≤Nal≤52.5%, for example 0%, 0.9%, 2.5%. The percentage refers to the quantity of the compound of formula (IA) based on potentiometric titration with a silver electrode according to the US Pharmacopeia monograph.

The composition of the present invention appears not to have been previously described and enables the problems previously mentioned to be overcome, enabling the necessary dose for diagnostic imaging to be prepared simultaneously.

The compound of formula (IB) used in the present invention is the one commercially available. In particular, the compound of formula (IA) of the present invention may be the one available on the market but it is conveniently prepared as described in the Italian patent application filed by the same Applicant (IT102021000006794), not yet published. The aforesaid application relates to a new process for preparing indocyanine green of formula (IA), with a total impurity content ≤0.5% and % and individual impurity ≤5 0.10%, purity determined by a new HPLC analytical method at the wavelength of 254 nm, and the related composition with stable NaI, soluble in water and with an NaI content ≤2.5%.

The process envisages the synthesis of the compound of formula (IA) comprising the following steps:
a. reacting the compound of formula (II) 1,1,2-trimethyl-1h-benzo[e]indole,

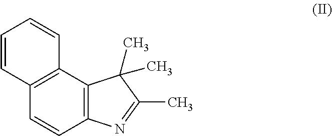 [SMILES: CC1=Nc2ccc3ccccc3c2C1(C)C]

with 1,4-butansultone of formula (III)

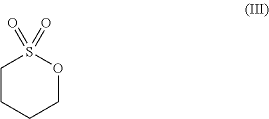

in an appropriate high boiling point solvent to provide 4-(1,1,2-trimethyl-1H-benzo[e]indolyl-3-il)butan-1-sulfonate of formula (IV), according to known methods;

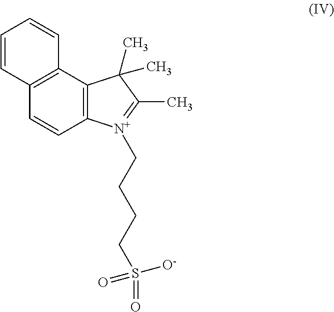 [SMILES: CC1=[N+](CCCCS(=O)(=O)[O-])c2ccc3ccccc3c2C1(C)C]

b. reacting the compound of formula (IV) with the compound of formula (V),

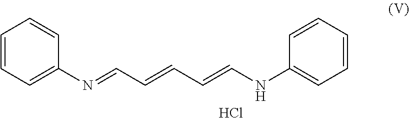 [SMILES: C(=C\C=N\c1ccccc1)/C=C/Nc1ccccc1 ; Cl]

benzenamine, N -[(2E,4E)-5-(phenylamino)-2,4-pentadien-1-ylidene]-, hydrochloride (1:1) (known as GAD), in the presence of acetic anhydride, sodium acetate and using a dipolar aprotic solvent to provide the final compound of formula (IA), without isolating any intermediate.

Step a) is also well known and the reaction can be carried out at a temperature that depends on the high boiling point solvent used such as, for example, the following aprotic solvents: hexane, cyclohexane, toluene, xylene, tetrahydrofuran, acetone, acetonitrile, 1,4-dioxane, diethyl ether, dichloromethane, ethyl acetate, N,N-dimethylformamide, methyl tert-butyl ether or the like, xylene and acetone.

The Applicant used xylene as a solvent at a temperature of around 130° C. Anisol can also be used with good results in terms of reaction speed, obtaining complete conversions in 7-8 hours at 140-150° C. instead of the usual 24 h necessary with xylene at 125-130° C. The intermediate compound of formula (IV) is isolated by precipitation adding acetone to the reaction mixture and is used as such, wet, without being recrystallized as described by US 2019/0337896.

The process is hence characterized by the direct performance of step b), i.e. “one step”, without isolating any intermediate and without the need to purify either the intermediate VI or the intermediate VII, as happened in synthesis already known in the state of the art and discussed previously.

Step b) is performed as already known by the condensation of the compound of formula (IV) with the compound of formula (V) in the presence of a solvent (acetonitrile), acetic anhydride and sodium acetate. The reaction is performed at a temperature comprised between 40-50° C. to form the crude compound of formula (IA). The compound of formula (V) and the compound of formula (IV) are dissolved in acetonitrile in the presence of sodium acetate (4 equivalents). The acetic anhydride (4 equivalents) is then added at the temperature that is lower than the one declared in US 2019/0337896 and reacted at the same temperature for a time comprised between 1-3 hours. The use of acetonitrile in this “One-Pot” step enabled the use of acetic anhydride in the minimum quantities for having a complete reaction, hence without using it as a reaction solvent. This made its elimination by distillation simpler and therefore the use of the water/isopropanol mixture then used for the isolation of the crude compound of formula (IA) safer.

In fact, the subsequent processing was carried out using isopropanol to separate the crude solid of the compound of formula (IA).

The compound of formula (IA) thus obtained in crude form is already in itself characterized by a high HPLC purity level (>90%) and the only significant impurity present is the impurity A. The compound (IA) can be conveniently purified by crystallization in isopropanol/H₂O rather than according to the known methods such as the methanol/isopropanol mixture of US 2019/0337896 or acetone, isopropanol, or methanol used in the other cited references. None of the references previously mentioned the use of the isopropanol/H₂O mixture in the appropriate ratios, chosen from the following: Isopropanol/Water: 5.9/3.4 or 7.4/3.4 or 9.9/3.4 expressed in volumes in litres/kg of the crude compound of formula (IA).

The use of isopropanol/H₂O has the advantage of providing the compound of formula (IA) at a purity ≥99.5 despite the fact that the intermediate (VI) has not been isolated.

The solid compositions in powder form of the compounds of formula (IA) and (IB) of the present invention are conveniently contained in relevant containers able to ensure the sterility of the composition and the absence of contact with air, especially with oxygen.

A further object of the present invention is a convenient “single dose” composition comprising, in particular, 5 mg of the compound of formula (IA) and 200 mg of the compound of formula (IB). Such composition is particularly advantageous for a specialist when it is necessary to prepare a mixture of the two components for being injected at the right dosage usually used in clinical practice for a single injection.

A further object of the present invention is a kit comprising the container comprising the composition according to the present invention and a vial of sterile water adapted to reconstitute the injectable solution.

An object of the present invention is also a kit comprising the solid composition of the present invention of the compounds of formula (IA) and (IB) contained in a relevant container which guarantees the sterility of the composition and does not come into contact with air and in particular oxygen, which can cause the known decomposition.

### Experimental Part

### Freeze-Drying Conditions

The freeze-drying conditions used for all the preparations are as follows:
Freeze-dryer: Edwards MINIFAST 680
Temperature: start of freeze-drying −40° C.; end of freeze-drying +5° C.
Pressure (Vacuum): start of freeze-drying 6.6*10² mbar, end of freeze-drying 4.6*10⁻² mbar
Freeze-drying time: 72 h
Amber glass vials insufflated with nitrogen and sealed at the end of distillation

### Analytical Method Used for Determining the Purity of the Compound of Formula (IA)

Column HPLC: ODS Hypersil 4.6×250 mm 5 μm
Column temperature: 40° C.
Detector: UV 254 nm
Step A: Ammonium Formate 4.09 g/L at pH=5.0 with Formic Acid
Step B: Acetonitrile
Mixture phase: 70:30 A:B
Flow rate: 1.5 ml/min
Injection volume: 10 μL
Analysis time: 30 minutes

Gradient:

| 0 minutes | 70% A | 30% B |
|---|---|---|
| 14 minutes | 25% | 75% |
| 22 minutes | 25% | 75% |
| 23 minutes | 70% | 30% |
| 30 minutes | 70% | 30% |

White: mixture phase

### Sample Preparation (Method 1)

40 mg in a 50 ml flask; dissolve and bring to the correct volume with the mixture phase.

Sonicate for 5 minutes verifying complete solubilization.

### Inject the Sample Straight Away as it is not Stable for Over 30 Minutes.

### Sample Preparation (Method 2)

40 mg in a 50 ml flask; dissolve and bring to the correct volume with methanol. Sonicate for 5 minutes verifying complete solubilization.

Inject immediately.

### Inject the Sample Straight Away as it is not Stable for Over 30 Minutes.

The use of methanol enables the compound of formula (IA) to be stabilized better, preventing the formation of a degradation impurity having an [MH]⁺: 752.5, i.e. “−1” with respect to the product.

Inject every 1-10 minutes.

### EXAMPLE 1

Preparation of 4-(1,1,2-trimethyl-1H-benzo[e]indolyl-3-il)butan-1-sulfonate of formula (IV)

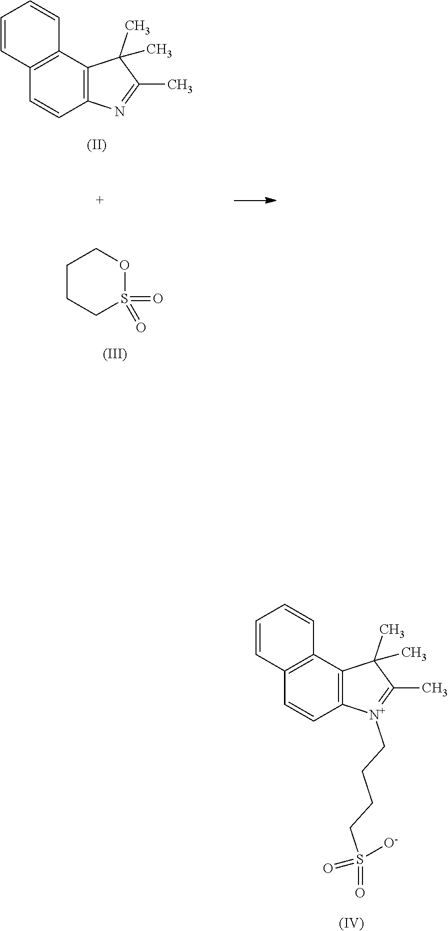

31.1 g of the compound of formula (II) (0.15 mol, 1 eq., commercially available), 40.5 g of the compound of formula (III) (0.30 mol, 2 eq., commercially available) in 93 ml of xylene are loaded into a 2 L reactor under a flow of nitrogen. The suspension is stirred and heated to the temperature of about 130° C. for 24 hours. The suspension is cooled and acetone (200 ml) is added. The solid obtained is then filtered and dried in a vacuum. Thus 48.5 g of the desired compound are obtained, corresponding to a 94.5% yield (HPLC purity: 97-98%). Alternatively, anisol can be used as the solvent, instead of xylene, using the same amount. Following the same protocol but carrying out the reaction at 140° C., the conversion of the product is completed in 6-8 hours. The yield and quantity that can be obtained are the same as in the reaction in xylene.

### EXAMPLE 2

### Preparation of the Compound of Formula (IA) (One-Pot Synthesis)

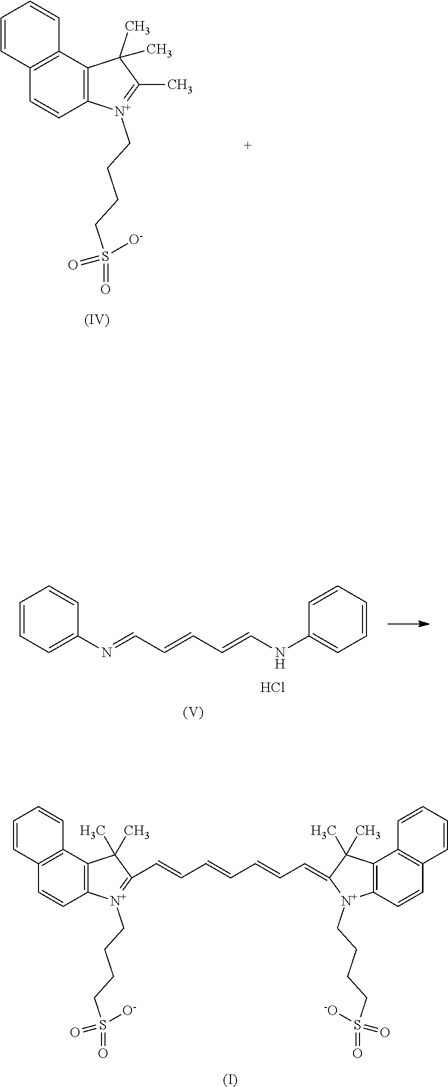

20.0 g of the compound of formula (V) (0.07 mol, 1 eq., commercially available), 48.5 g of the compound of formula (IV) (prepared as described in Example 1, 0.14 mol, 2 eq.), 23 g of sodium acetate (0.28 mol, 4 eq.) and 180 ml of acetonitrile are loaded into a 1 L reactor under a flow of nitrogen. The suspension is stirred at 20-25° C. and 28.8 g (0.28 mol, 4 eq.) of acetic anhydride are dripped in 5-10 minutes. The suspension is heated to a temperature between 45-50° C. and stirring continues for about 2 hours. The reaction mixture is concentrated in a vacuum keeping the temperature comprised between 4° and 50° C. It is then brought back to atmospheric pressure and 100 ml of iso-propanol are added then the reaction mixture is concentrated again in a vacuum, keeping the temperature between 4° and 50° C.

Then water (180 ml) and isopropanol (320 ml) are loaded, the product is put into solution at 50-55° C., then isopropanol (100 ml) is added and the mixture is cooled gradually to 20-25° C.

It is filtered and washed with isopropanol.

Thus the wet desired compound is obtained (yield according to the weight loss: 46.3 g, 85.1% with respect to compound (V) with HPLC purity 80-85%.

### EXAMPLE 3

Purification of the Compound of Formula (IA) (without NaI)

49 g of indocyanine green wet with isopropanol (prepared as described in Example 2, equal to 24.4 g dry), 130 ml of isopropanol and 77 ml of water are loaded into a 1 litre flask. It is heated to 50-55° C. and stirred until complete solubilization. The pH of the solution is corrected to 7.5-8.5 with 5% NaOH and it is cooled to 40-45° C.

Maintaining 40-45° C., isopropanol (48 ml) is added, then it is cooled gradually to 20-25° C., stirring continues for 1.5 h, then it is filtered and washed with isopropanol (2×48 ml).

The powder is dried in a vacuum at 60° C. for 40 hours.
Yield: 20 g (82.9%).
HPLC purity: 99.5%; impurity A: 0.40%.
Sodium iodide content: 0%.

### EXAMPLE 4

Preparation of the Compound of Formula (IA) with NaI (I Crystallization)

93.6 g of the crude compound of formula (IA) (46.3 g of theoretical dry compound, prepared as described in Example 2) and sodium iodide (1.39 g; 3% w/w) are suspended in 250 ml of isopropanol and in 148 ml of water. The suspension is heated to a temperature of 55-60° C. and stirred until complete dissolution. The pH is corrected to 7.5-8.5 using 2.5% w/w sodium hydroxide solution. The solution is cooled to a temperature comprised between 45-50° C. and in 15-30 minutes 93 ml of isopropanol are added. It is cooled slowly until reaching the temperature of 20-25° C. and stirring continues for 30 minutes. The suspension is then brought to 35-40° C., stirred for about 1 hour, then cooled again in about 2 h to 20-25° C. and finally filtered at 20-30° C. and washed with isopropanol.

It is dried in a vacuum at 50-80° C. for 8-48 hours and 35.89 g of the desired product are obtained with a yield of 77.5% (with respect to the respective crude dry product loaded).
HPLC purity: 99.6%; impurity A: 0.28%.
Iodide (potentiometric titration with silver electrode): 1%

In the event in which the known impurities are >0.15% and the unknown ones >0.1%, it is possible to perform a second crystallization using less sodium iodide, which is essential for keeping the quantity of sodium iodide in the finished product less than 2.5% (example 6).

### EXAMPLE 5

Preparation of the Compound of Formula (IA) with NaI (II Crystallization)

The compound of formula (IA), wet, obtained from the first crystallization (prepared as described in example 4) (63.7%, equal to 35.9 g of corresponding dry product based on weight loss), sodium iodide (0.54 g; 1.5% w/w), isopropanol (194 ml) and water (115 ml) are loaded into a 1 litre reactor. It is heated to 55-60° C. until complete dissolution, then the solution is filtered on cardboard and the filter is washed with water (7 ml) and then with isopropanol (18 ml). The filtrate is brought to 55-60° C., if necessary the pH is corrected with NaOH diluted in the range 7.5-8.5, then cooled to 45-50° C. and isopropanol (54 ml) is added in about 30 minutes.

It is cooled slowly to 20-25° C., heated again to 35-40° C. for about 1 hour, then brought back to 20-25° C. in about 1 hour and stirring continues for 30 minutes.

The suspension is filtered and washed with isopropanol, obtaining the wet product which is dried in a vacuum at 50-80° C. for 24-48 hours.
Yield: 26.9 g (75%).

The purity of the product thus obtained measured using the HPLC method of the invention is 99.5%.
HPLC purity: 99.92%
Impurity A, B, C, D, E and F: non quantifiable (HPLC);
Maximum unknown impurity: 0.084% (HPLC; rrt: 0.45).
Sodium iodide (potentiometric titration according to USP monograph): 0.9%.
Residual isopropanol: 1597 ppm.

### EXAMPLE 5

### Dry-Freezing Tests on the Compounds of Formula (IA) and (IB)

Initially, dry-freezing tests were carried out on 10 ml aqueous solutions of the compound (IA) prepared as described in example 5 at the concentration of 5 mg/ml_{water} containing 0.9% of sodium iodide, of the compound of formula (IA) prepared as described in Example 3 at the concentration of 5 mg/ml_{water} prepared without sodium iodide and of the compound of formula (IB), commercially available, at the concentration of 200 mg/ml_{water}.

The dry-freezing was completed in 72 hours and all three dry frozen products were soluble in water.

In particular, the compound of formula (IA) obtained without the use of sodium iodide (sodium iodide by potentiometric titration with silver electrode=0%) is not soluble in water at 20-25° C. at the clinical use concentration of 5 mg/ml or 2.5 mg/ml if used as such, as a dry isolated powder after crystallization.

Surprisingly, the same freeze-dried powder is instead soluble at the concentrations of 2.5 mg/ml, 5 mg/ml and also 10 mg/ml.

The solubility was evaluated by filtering all the solution obtained on a syringe filter provided with 0.45 μm holes and for the 5 mg/ml concentration of freeze-dried powders, with or without NaI, also 0.2 μm holes, observing that the filtration takes place fluidly, without any residue remaining either on the filter or in the vial from which the solution was withdrawn.

The molecule is probably soluble in itself, but due to kinetic reasons it does not dissolve in reasonable time scales. As the dry frozen powder, on the other hand, has a high relative surface area of contact with water, it tends to dissolve completely and immediately, without even needing to be sonicated. These results are summarised in Table 1:

TABLE 1

| | | | | Filtration on 0.45 μm | | | Filtration on 0.20 μm | | |
|---|---|---|---|---|---|---|---|---|---|
| | | | | syringe filter | | | syringe filter | | |
| | | Freeze- | | ml | | | ml | | |
| Batch | NaI | dried | mg/ml | filtered | Pressure | Filter | filtered | Pressure | Filter |

| | | | | | | |
|---|---|---|---|---|---|---|
| 069/079 | 0 | NO | 10 | 0 | Block | nd |
| | | NO | 5 | 0 | Block | nd |
| | | NO | 2.5 | 0 | Block | nd |
| | | Yes | 10 | 10 | Low | nd |
| | | Yes | 5 | 10 | Low | nd |
| | | Yes | 2.5 | 10 | Low | nd |
| 065/068 | 0.90% | NO | 10 | 2 | Block | nd |
| | | NO | 5 | 10 | High | nd |
| | | NO | 2.5 | 10 | Low | nd |
| | | Yes | 10 | 10 | Low | nd |
| | | Yes | 5 | 10 | Low | nd |
| | | Yes | 2.5 | 10 | Low | nd |

Nd: no lump identified on the filter

Surprisingly it was also found that the two components can be freeze-dried simultaneously dosing them in the appropriate quantities into the same vial. The freeze-dried product thus obtained is also soluble in water at low concentrations of sodium iodide and therefore suitable for the simultaneous administration of the two dyes by injection once the aqueous solution has been reconstituted.

The solubilization of the freeze-dried formulations from 13 to 18 (table 2) is also immediate, regardless of the presence of NaI or not.

In particular, it is possible to prepare the freeze-dried products containing the quantities of the compound of formula (IA), the compound of formula (IB) and sodium iodide (expressed as a % weight with respect to the compound of formula (IA) reported in the following Table 2:

TABLE 2

| | Formulation | | | | | | |
|---|---|---|---|---|---|---|---|
| | | Indocyanine | NaI | | | Conc. | Conc. |
| | Sodium | green with | added | Solut. | | Sodium | Indocyanine |
| | fluorescein | 0.9% iodide | % W vs | NaI | Water | fluorescein | green |
| Vial* | mg | mg | ICG | ml | ml | mg/ml¹ | mg/ml² |
| 1 | 2500 | 100 | 1.5 | 5 | 5 | 250 | 10 |
| 2 | 1250 | 50 | 1.5 | 2.5 | 2.5 | 250 | 10 |
| 3 | 2500 | 50 | 1.5 | 2.5 | 7.5 | 250 | 5 |
| 4 | 1250 | 25 | 1.5 | 1.25 | 3.75 | 250 | 5 |
| 5 | 2000 | 50 | 1.5 | 2.5 | 7.5 | 200 | 5 |
| 6 | 1000 | 25 | 1.5 | 1.25 | 3.75 | 200 | 5 |
| 7 | 2000 | 50 | 0 | 0 | 10 | 200 | 5 |
| 8 | 1000 | 25 | 0 | 0 | 5 | 200 | 5 |
| 11³ | 200 | 5 | 0 | 0 | 5 | 40 | 1 |
| 13^{4.5} | 200 | 5 | 0 | 0 | 5 | 40 | 1 |
| 17^{4.5} | 100 | 2.5 | 0 | 0 | 5 | 20 | 0.5 |
| 15^{4.5} | 400 | 10 | 0 | 0 | 5 | 80 | 2 |
| | | Indocyanine | | | | | |
| | Sodium | green | | | | | |
| | fluorescein | without NaI | | | | | |
| 9 | 2000 | 50 | 0 | 0 | 10 | 200 | 5 |
| 10 | 1000 | 25 | 0 | 0 | 5 | 200 | 5 |
| 12 | 200 | 5 | 0 | 0 | 5 | 40 | 1 |
| 14⁵ | 200 | 5 | 0 | 0 | 5 | 40 | 1 |
| 18⁵ | 100 | 2.5 | 0 | 0 | 5 | 20 | 0.5 |
| 16⁵ | 400 | 10 | 0 | 0 | 5 | 80 | 2 |

¹Amber glass vial placed under nitrogen after freeze-drying
²ml of solvent (water + additive solution)
³NaI in ICG: 1.3%
⁴NaI in ICG: 2.1%
⁵solutions filtered on 0.2 μm filter directly in the vial before freeze-drying

### EXAMPLE 6

### Preparation of Dry Frozen Product of the Compound of Formula (IB)

The compound of formula (IB), sodium fluorescein (commercially available, 5.0 g) is dissolved in water (25 ml) and sonicated for 1 minute. This solution is used to fill 5 different amber glass vials (about 5 ml of solution per vial). The vials are freeze-dried using the freeze-drying conditions already described.

### EXAMPLE 7

### Preparation of Dry Frozen Product of the Compound of Formula (IA)

The compound of formula (IA) (prepared according to example 5 or example 3, 125 mg) is dissolved in water (25 ml) and sonicated for 1 minute.

This solution is used to fill 5 different amber glass vials (about 5 ml of solution per vial). The vials are freeze-dried using the freeze-drying conditions already described.

### EXAMPLE 8

### Preparation of the Solid Composition of the Compounds of Formula (IA) and (IB)

Preparation of the NaI solution

In a 100 ml graduated flask, weigh 30 mg of sodium iodide and bring up to volume with water for HPLC. Sonicate for 5 minutes.

General method for preparing mixtures of the compounds of formula (IA) and (IB).

The exact quantities of every component of the mixture are provided in table 2.

In a 20 ml amber glass vial accurately weigh the compound of formula (IA) or (IB).

Add the correct volume of the sodium iodide solution previously prepared.

Add the correct volume of water.

Sonicate the vials for about 1 minute under nitrogen at 20-25° C.

Freeze-dry using the freeze-drying conditions already described.

For every solution, 5 replicas were prepared.

### EXAMPLE 9

### General Method for Preparing Mixtures of the Compounds of Formula (IA) and (IB): Formulations 11, 12, 13, 14, 15, 16, 17, 18

The exact quantities of every component of the mixture are provided in table 2.

In a 10 ml flask accurately weigh the compound of formula (IA) or (IB).

Add the correct volume of water.

Sonicate the vials for about 1 minute under nitrogen at 20-30° C.

Where envisaged, filter the entire solution thus obtained on a 0.2 um syringe filter inside a new vial.

Freeze-dry using the freeze-drying conditions previously described.

### EXAMPLE 10

Stability of the solid composition of the compounds of formula (IA) and (IB) The compound of formula (IA) (with or without iodide) and the compound of formula (IB) were analysed in HPLC according to the following method as such and after freeze-drying. The analysis demonstrates that such compounds are stable.

### Analytical Method (HPLC)

Column HPLC: ODS Hypersil 4.6×250 mm 5 μm
Column temperature: 40° C.
Detector: UV 254 nm
   Step A: Ammonium Formate 4.09 g/L at pH=5.0 with Formic Acid

### Step B: Acetonitrile

Mixture phase: 70:30 A:B
Flow rate: 1.5 ml/min
Injection volume: 10 μL
Analysis time: 30 minutes

Gradient:

| 0 minutes | 70% A | 30% B |
|---|---|---|
| 14 minutes | 25% | 75% |
| 22 minutes | 25% | 75% |
| 23 minutes | 70% | 30% |
| 30 minutes | 70% | 30% |

The formulations from 13 to 18 and the compounds used for the preparation thereof were analysed with the following analytical method. The analysis confirms that the purity of IA >99.5% injected as such and the stability of the formulation after freeze-drying, evaluated based on the fact that there is no other peak in the chromatogram except for Ia and Ib.

### Analytical Method (HPLC)

Column HPLC: Polaris 3 C18-A 150×4.6 mm
Column temperature: 20° C.
Detector: UV 254 nm
Step A: Ammonium acetate 2.3 g in 1000 ml brought to pH 6.8±0.05 with diluted acetic acid or ammonia
Step B: Acetonitrile
Diluent: methanol
Flow rate: 1.5 ml/min
Injection volume: 10 μL
Analysis time: 34 minutes
Autosampler temperature: 5° C.

Gradient:

| 0 minutes | 70% A | 30% B |
|---|---|---|
| 24 minutes | 40% | 60% |
| 28 minutes | 40% | 60% |
| 29 minutes | 70% | 30% |
| 34 minutes | 70% | 30% |

Sample solution: 1.5 mg/ml in methanol. Inject immediately after preparing the solution. The mixtures of formula (IA) and (IB) described in Table 2 were prepared and then appropriately diluted and injected in HPLC to evaluate the profile at T₀.

The same mixtures were re-prepared exactly as described in the experimental part, dry frozen and controlled in HPLC after freeze-drying, 7 days later. During these 7 days the samples were stored inside their amber glass vials in a protective atmosphere at room temperature. The freeze-dried products were dissolved in water at the same dilution as the not freeze-dried samples.

From this analysis it is clear that freeze-drying does not degrade the compound of formula (IA) either as such or in mixture with the compound of formula (IB), with or without sodium iodide. The stability of the freeze-dried powders stored in the vials was evaluated using HPLC even after 6 months. The freeze-dried products are stable, even without the presence of sodium iodide, formulation 9.

The reconstituted solution was stable if stored at 4-10° C. for at least 24 hours (formulations 1, 3, 5 and 7).

| | HPLC (A %) | | | |
|---|---|---|---|---|
| Formulation | | | | Total |
| 1 | Fluo | ICG | Fluo + ICG | impurities |
| Not freeze-dried | 94.6 | 5.1 | 99.7 | 0.3 |
| Freeze-dried | 95.3 | 4.5 | 99.8 | 0.2 |
| Freeze-dried after 6 months | 94.7 | 5 | 99.7 | 0.3 |
| Freeze-dried solution reconstituted | 94.4 | 5.1 | 99.5 | 0.5 |
| after 24 hours at +4° C. | | | | |

| | HPLC (A %) | | | |
|---|---|---|---|---|
| Formulation | | | | Total |
| 3 | Fluo | ICG | Fluo + ICG | impurities |
| Not freeze-dried | 97.5 | 2.2 | 99.7 | 0.3 |
| Freeze-dried | 97.5 | 2.2 | 99.7 | 0.3 |
| Freeze-dried after 6 months | 97.2 | 2.6 | 99.8 | 0.2 |
| Freeze-dried solution reconstituted | 97.1 | 2.4 | 99.5 | 0.5 |
| after 24 hours at +4° C. | | | | |

| | HPLC (A %) | | | |
|---|---|---|---|---|
| Formulation | | | | Total |
| 5 | Fluo | ICG | Fluo + ICG | impurities |
| Not freeze-dried | 96.9 | 2.6 | 99.5 | 0.5 |
| Freeze-dried | 97.5 | 2.2 | 99.7 | 0.3 |
| Freeze-dried after 6 months | 96.7 | 3.1 | 99.8 | 0.2 |
| Freeze-dried solution reconstituted | 96.4 | 3.1 | 99.5 | 0.5 |
| after 24 hours at +4° C. | | | | |

| | HPLC (A %) | | | |
|---|---|---|---|---|
| Formulation | | | | Total |
| 7 | Fluo | ICG | Fluo + ICG | impurities |
| Not freeze-dried | 97.4 | 2.4 | 99.8 | 0.2 |
| Freeze-dried | 97.1 | 2.5 | 99.6 | 0.4 |
| Freeze-dried solution reconstituted | 96.4 | 3.1 | 99.5 | 0.5 |
| after 24 hours at +4° C. | | | | |

| | HPLC (A %) | | | |
|---|---|---|---|---|
| Formulation | | | | Total |
| 8 | Fluo | ICG | Fluo + ICG | impurities |
| Not freeze-dried | 97.1 | 2.6 | 99.7 | 0.3 |
| Freeze-dried | 94.5 | 5.3 | 99.8 | 0.2 |
| Freeze-dried after 6 months | 96.5 | 3.2 | 99.7 | 0.3 |

| | HPLC (A %) | | | |
|---|---|---|---|---|
| Formulation | | | | Total |
| 9 | Fluo | ICG | Fluo + ICG | impurities |
| Not freeze-dried | 98.1 | 1.3 | 99.4 | 0.6 |
| Freeze-dried | 97.2 | 2.5 | 99.7 | 0.3 |
| Freeze-dried after 6 months | 96.5 | 3.2 | 99.7 | 0.3 |

The invention claimed is:

1. Solid composition of indocyanine green of formula (IA) and sodium fluorescein of formula (IB).

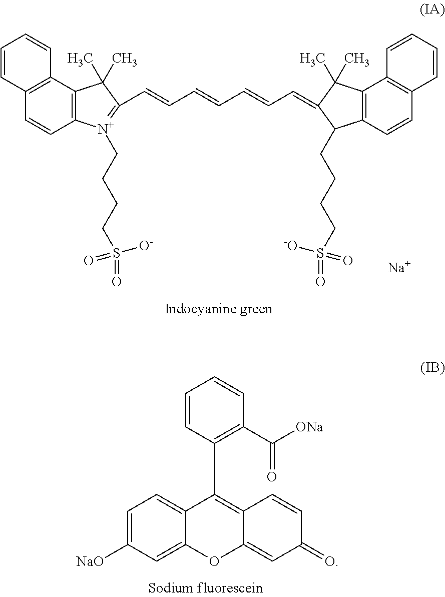

2. The solid composition according to claim 1, wherein the amount of indocyanine green is 2.5 mg, 5 mg, 10 mg, 25 mg, 50 mg or 100 mg and the amount of sodium fluorescein is 0.1 g, 0.2 g, 0.4 g, 2.5 g, 2.0 g, 1.25 g or 1 g.

3. The solid composition according to claim 1, comprising an amount of NaI equal to 0%≤NaI≤2.5%, with respect to the amount of the compound (IA) based on a potentiometric title with silver electrode in accordance with USP monograph.

4. The solid composition according to claim 1, wherein the amount of indocyanine green is 5 mg and the amount of sodium fluorescein is 0.2 g.

5. Process for preparing the solid composition of claim 1, said process comprising
lyophilizing an aqueous solution formed by the compounds of formula (IA) and (IB).

6. The process according to claim 5, wherein the aqueous solution is microfiltered before the lyophilizing step.

7. Method of diagnostic imaging in ophthalmology in a subject in need thereof with the composition according to claim 1, said method comprising
administering to said subject an effective amount of said composition.

8. Kit comprising the solid composition according to claim 1 and a vial of sterile water suitable for the production of an intravenous injection solution for use in imaging in ophthalmology.
